(19) United States
(12) Patent Application Publication
(10) Pub. No.: US 2025/0014678 A1
(43) Pub. Date: Jan. 9, 2025

(54) METHODS AND SYSTEMS FOR REPORTING CLINICALLY-ACTIONABLE POTENTIAL GERMLINE PATHOGENIC VARIANT SEQUENCES

(71) Applicant: Foundation Medicine, Inc., Boston, MA (US)

(72) Inventors: Kali Chatham DOUGHERTY, Cambridge, MA (US); Erica GORNSTEIN, Cambridge, MA (US); Kimberly MCGREGOR, Grand Junction, CO (US); Rachel ERBACH, Cambridge, MA (US); Garrett FRAMPTON, Cambridge, MA (US)

(73) Assignee: Foundation Medicine, Inc., Boston, MA (US)

(21) Appl. No.: 18/712,646

(22) PCT Filed: Nov. 23, 2021
(86) PCT No.: PCT/US2021/072577

§ 371 Date: May 22, 2024

(51) Int. Cl. G16B 20/20; C12Q 1/6886; G16H 50/20

(52) U.S. Cl. CPC G16B 20/20; C12Q 1/6886; G16H 50/20; C12Q 2600/156

(57) ABSTRACT

Methods for reporting clinically-actionable potential germline pathogenic variants identified in nucleic acid sequencing data are described. The methods may comprise, for example, receiving variant sequence data for a sample from a subject; filtering the variant sequence data to identify variant sequences located in a gene locus known to comprise clinically-actionable germline variants; filtering the variant sequences identified as being located in such a gene locus to identify those that are potentially germline pathogenic; filtering the resulting list of potentially germline pathogenic variant sequences to identify those that have an allele frequency greater than a first predetermined threshold; and based on a determination that a potentially germline pathogenic variant sequence has an allele frequency greater than the first predetermined threshold, reporting the variant sequence as a clinically-actionable potential germline pathogenic variant.

### FIELD OF THE INVENTION

The present disclosure relates generally to methods and systems for analyzing genomic profiling data, and more specifically to methods and systems for reporting clinically-actionable potential germline pathogenic variant sequences identified in genomic profiling data.

### BACKGROUND

Advances in nucleic acid sequencing methodology and bioinformatics analysis techniques have enabled corresponding advances in the identification of variant sequences in genomic profiling data for a patient or other subject that may have diagnostic and/or prognostic implications for disease states such as cancer, and which may in turn influence healthcare decisions and impact healthcare outcomes for the patient. Examples of variant sequences (or mutations) include single nucleotide variants (e.g., point mutations), short variants (e.g., genomic alterations such as insertions or deletions of less than about 50 base pairs in length), or structural variants (e.g., genomic alterations such as longer insertions, deletions, duplications, inversions, and translocations of greater that about 50 bp in length). In multicellular organisms comprising dedicated reproductive cells (gametes), such variants can generally be classified as either somatic or germline mutations. Somatic mutations occur in non-gamete cells and cannot be inherited-only tissues derived directly from the mutated cell(s) are affected. Germline mutations occur in gamete cells and can thus be inherited by offspring. As a result, germline mutations are present in every cell of a descendant.

Recently, methods have been developed for sequencing, e.g., tumor samples, to identify variant sequences without using a paired/matched normal sample, which is often unavailable and which, even if available, requires additional sample processing and sequencing steps which add to the overall cost of a diagnostic or prognostic procedure. Such methods may detect and report both somatic and germline variants, but often cannot distinguish between them.

Potential germline variants may be found in patients who do not have a clinical indication for performing germline testing, such as a family history or a specific cancer type, or who were missed by routine and/or guideline-based screening methods. Detection and reporting of potential pathogenic germline variants, along with a recommendation for performing follow-up germline testing, can have important implications for both the affected patient and their family members. Thus there remains a need for methods to accurately, efficiently, and effectively identify and report potential germline pathogenic variants detected in nucleic acid sequencing data.

### BRIEF SUMMARY OF THE INVENTION

Disclosed herein are methods and systems for identifying and reporting clinically-actionable potential germline variants. The methods comprise identifying variant sequences (e.g., short variants) in sequence read data for selected gene loci (e.g., cancer susceptibility gene loci) that may represent actionable pathogenic germline variants, and reporting them to ensure that the healthcare provider and patient consider follow-up germline testing and/or genetic counseling. Potential germline pathogenic variants are identified by first filtering the variant sequences occurring within one of the selected gene loci for inclusion in a genomic database (e.g., the ClinVar database, National Center for Biotechnology Information, Bethesda, MD) as being pathogenic or likely pathogenic, and then filtering the results by variant allele frequency (VAF) based on a comparison to a specified assay-specific VAF threshold.

Disclosed herein are methods comprising: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, sequence read data for the plurality of sequence reads; identifying, using the one or more processors, variant sequence data for one or more gene loci within the sequence read data for the plurality of sequence reads; filtering, using the one or more processors, the variant sequence data to identify variant sequences located in a gene locus from a list of gene loci comprising clinically-actionable germline variants; filtering, using the one or more processors, the variant sequences located in a gene locus on the list to identify variant sequences that are potentially germline pathogenic based on a sequence comparison to known clinically-actionable germline variants for the gene locus; filtering, using the one or more processors, the potentially germline pathogenic variant sequences to identify variant sequences that have an allele frequency greater than a first predetermined threshold; and based on a determination that a particular potentially germline pathogenic variant sequence has an allele frequency greater than the first predetermined threshold, identifying the variant sequence as a clinically-actionable potential germline pathogenic variant.

In some embodiments, the method further comprises generating a report that identifies the variant sequence as a clinically-actionable potential germline pathogenic variant. In some embodiments, the method further comprises generating a recommendation for follow-up germline testing if a variant sequence is identified as being a clinically-actionable potential germline pathogenic variant, and including the recommendation in the report. In some embodiments, the method further comprises providing a description of one or more potential disease states that may be associated with the clinically-actionable potential germline pathogenic variant as part of the report. In some embodiments, the method further comprises providing a description of one or more treatment options that may be applicable for the one or more potential disease states as part of the report. In some embodiments, the method further comprises providing a description of potential disease state prevention measures for the subject or the subject's family members as part of the report. In some embodiments, the list of gene loci comprises a list of cancer susceptibility genes that comprise clinically-actionable germline variants. In some embodiments, the list of gene loci comprises the ATM, BAP1, BRCA1, BRCA2, BRIP1, CHEK2, FH, FLCN, MLH1, MSH2, MSH6, MUTYH, PALB2, PMS2, POLE, RAD51C, RAD51D, RET, SDHA, SDHB, SDHC, SDHD, TSC2, VHL gene loci, or any combination thereof. In some embodiments, the subject is suspected of having or is determined to have cancer.

In some embodiments, the method further comprises obtaining the sample from the subject. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample. In some embodiments, the one or more adapters comprise amplification primers, flow cell adaptor sequences, substrate adapter sequences, or sample index sequences. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencer comprises a next generation sequencer. In some embodiments, one or more of the plurality of sequencing reads overlap one or more gene loci within one or more subgenomic intervals within the sample. In some embodiments, the method further comprises generating, by the one or more processors, a report comprising a list of one or more clinically-actionable potential germline pathogenic variants. In some embodiments, the method further comprises transmitting the report to a healthcare provider. In some embodiments, the report is transmitted via a computer network or a peer-to-peer connection.

Also disclosed herein are methods for reporting clinically-actionable potential germline variants comprising: receiving, at one or more processors, variant sequence data for one or more gene loci in a sample from a subject; filtering, using the one or more processors, the variant sequence data to identify variant sequences located in a gene locus from a list of gene loci comprising clinically-actionable germline variants; filtering, using the one or more processors, the variant sequences located in a gene locus on the list to identify variant sequences that are potentially germline pathogenic based on a sequence comparison to known clinically-actionable germline variants for the gene locus; filtering, using the one or more processors, the potentially germline pathogenic variant sequences to identify variant sequences that have an allele frequency greater than a first predetermined threshold; and based on a determination that a particular potentially pathogenic germline variant sequence has an allele frequency above the first predetermined threshold, reporting the potentially pathogenic germline variant sequence as a clinically-actionable potential germline pathogenic variant.

In some embodiments, the method further comprises generating a report that identifies the variant sequence as a clinically-actionable potential germline pathogenic variant. In some embodiments, the method further comprises generating a recommendation for follow-up germline testing if a variant sequence is reported as being a clinically-actionable potential germline pathogenic variant, and including the recommendation in the report. In some embodiments, the method further comprises providing a description of one or more potential disease states that may be associated with the clinically-actionable potential germline pathogenic variant as part of the report. In some embodiments, the method further comprises providing a description of one or more treatment options that may be applicable for the one or more potential disease states as part of the report. In some embodiments, the method further comprises providing a description of potential disease state prevention measures for the subject or the subject's family members.

In some embodiments, the list of gene loci is selected from a source of genomic information. In some embodiments, the source of genomic information comprises a public or private genomic database. In some embodiments, the list of gene loci comprises a list of cancer susceptibility genes that comprise clinically-actionable germline variants. In some embodiments, the list of cancer susceptibility genes comprises a list of gene loci for which variant sequences have a probability of greater than a second predetermined threshold of being germline in origin if identified during tumor sample sequence. In some embodiments, the second predetermined threshold ranges from 5% to 20%. In some embodiments, the list of gene loci comprises the ATM, BAP1, BRCA1, BRCA2, BRIP1, CHEK2, FH, FLCN, MLH1, MSH2, MSH6, MUTYH, PALB2, PMS2, POLE, RAD51C, RAD51D, RET, SDHA, SDHB, SDHC, SDHD, TSC2, VHL gene loci, or any combination thereof.

In some embodiments, the method further comprises identifying and reporting germline passenger mutations associated with a clinically-actionable potential germline pathogenic variants.

In some embodiments, the known clinically-actionable germline variants for the gene locus comprise variant sequences that have been observed previously as being germline in origin and are listed as either pathogenic or likely pathogenic in a public or private genomic database. In some embodiments, the variant sequences that have been observed previously as being germline in origin and are listed as either pathogenic or likely pathogenic have been submitted to the public or private genomic database by at least two submitters.

In some embodiments, the first predetermined threshold is determined empirically by calculating allele frequency values for variant sequence data comprising a known number of common germline variant sequences. In some embodiments, the first predetermined threshold is adjusted so that at least 85%, at least 90%, at least 95%, or at least 98% of the known germline variant sequences in the variant sequence data are reported as potential germline variants. In some embodiments, the first predetermined threshold is different for variant sequence data derived from different assay formats. In some embodiments, the first predetermined threshold for variant sequence data derived from a liquid biopsy assay format is larger than that for variant sequence data derived from a tissue-based assay format. In some embodiments, the first predetermined threshold ranges from about 5% to about 40%.

In some embodiments, the list of gene loci further comprises a list of gene loci comprising founder mutations associated with a disease state that may be the same as or different from a known or suspected disease state for the sample.

In some embodiments, the method further comprises testing the subject to confirm that the clinically-actionable potential germline pathogenic variant is a clinically-actionable germline pathogenic variant. In some embodiments, the method further comprises using the confirmed clinically actionable germline pathogenic variant to diagnose or confirm a diagnosis of disease or syndrome in the subject. In some embodiments, the disease is cancer. In some embodiments, the cancer is a hereditary cancer. In some embodiments, the syndrome is a predisposition syndrome.

In some embodiments, the method further comprises selecting an anti-cancer therapy to administer to the subject based on the confirmed clinically-actionable germline pathogenic variant. In some embodiments, the method further comprises determining an effective amount of the anti-cancer therapy to administer to the subject. In some embodiments, the method further comprises administering the anti-cancer therapy to the subject. In some embodiments, the anti-cancer therapy comprises chemotherapy, radiation therapy, immunotherapy, a targeted therapy, or surgery. In some embodiments, the cancer is a solid tumor. In some embodiments, the cancer is a hematological cancer. In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the method further comprises sequencing, by a sequencer, nucleic acid molecules derived from the sample to obtain a plurality of sequence reads, wherein one or more of the plurality of sequencing reads overlap one or more gene loci within one or more subgenomic intervals within the sample. In some embodiments, the method further comprising identifying variant sequence data for the one or more gene loci based on the plurality of sequence reads.

In some embodiments, the method further comprises generating a genomic profile for the subject that includes a list of one or more reported clinically-actionable potential germline pathogenic variants. In some embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the subject further comprises results from a nucleic acid sequencing-based test. In some embodiments, the method further comprises selecting an anti-cancer agent, administering an anti-cancer agent, or applying an anti-cancer treatment to the subject based on the generated genomic profile.

Disclosed herein are systems comprising: one or more processors; and a memory communicatively coupled to the one or more processors and configured to store instructions that, when executed by the one or more processors, cause the system to: receive variant sequence data for one or more gene loci within one or more subgenomic intervals in a sample from a subject; filter the variant sequence data to identify variant sequences located in a gene locus from a list of gene loci comprising clinically-actionable germline variants; filter the variant sequences located in a gene locus on the list to identify variant sequences that are potentially germline pathogenic based on a sequence comparison to known clinically-actionable germline variants for the gene locus; filter the potentially germline pathogenic variant sequences to identify variant sequences that have an allele frequency greater than a first predetermined threshold; and based on a determination that a particular potentially pathogenic germline variant sequence has an allele frequency greater above the first predetermined threshold, report the potentially pathogenic germline variant sequence as a clinically-actionable potential germline pathogenic variant.

In some embodiments, the instructions further comprise causing the system to make a recommendation for follow-up germline testing if a variant sequence is reported as being a clinically-actionable potential germline pathogenic variant. In some embodiments, the instructions further comprise instructions configured to generate a description of one or more potential disease states that may be associated with the clinically-actionable potential germline pathogenic variant as part of the report. In some embodiments, the instructions further comprise instructions configured to generate a description of one or more treatment options that may be applicable for the one or more potential disease states as part of the report. In some embodiments, the instructions further comprise instructions configured to generate a description of potential disease state prevention measures for the subject or the subject's family members. In some embodiments, the known clinically-actionable germline variants for the gene locus comprise variant sequences that have been observed previously as being germline in origin and are listed as either pathogenic or likely pathogenic in a public or private genomic database. In some embodiments, the variant sequences that have been observed previously as being germline in origin and are listed as either pathogenic or likely pathogenic have been submitted to the public or private genomic database by at least two submitters. In some embodiments, the first predetermined threshold is determined empirically by calculating allele frequency values for variant sequence data comprising a known number of common germline variant sequences. In some embodiments, the first predetermined threshold is adjusted so that at least 85%, at least 90%, at least 95%, or at least 98% of the known germline variant sequences in the variant sequence data are reported as potential germline variants. In some embodiments, the first predetermined threshold is different for variant sequence data derived from different assay formats. In some embodiments, the first predetermined threshold for variant sequence data derived from a liquid biopsy assay format is larger than that for variant sequence data derived from a tissue-based assay format. In some embodiments, the first predetermined threshold ranges from about 5% to about 40%. In some embodiments, the list of gene loci further comprises a list of gene loci comprising founder mutations associated with a disease state that may be the same as or different from a known or suspected disease state for the sample. In some embodiments, the report further comprises a recommendation for further testing of the subject to confirm that the clinically-actionable potential germline pathogenic variant is a clinically-actionable germline pathogenic variant. In some embodiments, the confirmed clinically actionable germline pathogenic variant is used to diagnose or confirm a diagnosis of disease or syndrome in the subject.

Disclosed herein are non-transitory computer-readable storage medium storing one or more programs, the one or more programs comprising instructions, which when executed by one or more processors of a system, cause the system to: receive variant sequence data for one or more gene loci within one or more subgenomic intervals in a sample from a subject; filter the variant sequence data to identify variant sequences located in a gene locus from a list of gene loci comprising clinically-actionable germline variants; filter the variant sequences located in a gene locus on the list to identify variant sequences that are potentially germline pathogenic based on a sequence comparison to known clinically-actionable germline variants for the gene locus; filter the potentially germline pathogenic variant sequences to identify variant sequences that have an allele frequency greater than a first predetermined threshold; and based on a determination that a particular potentially pathogenic germline variant sequence has an allele frequency greater above the first predetermined threshold, report the potentially pathogenic germline variant sequence as a clinically-actionable potential germline pathogenic variant.

In some embodiments, the instructions further comprise instructions configured to generate a recommendation for follow-up germline testing if a variant sequence is reported as being a clinically-actionable potential germline pathogenic variant. In some embodiments, the instructions further comprise instructions configured to generate a description of one or more potential disease states that may be associated with the clinically-actionable potential germline pathogenic variant as part of the report. In some embodiments, the instructions further comprise instructions configured to generate a description of one or more treatment options that may be applicable for the one or more potential disease states as part of the report. In some embodiments, the instructions further comprise instructions configured to generate a description of potential disease state prevention measures for the subject or the subject's family members. In some embodiments, the known clinically-actionable germline variants for the gene locus comprise variant sequences that have been observed previously as being germline in origin and are listed as either pathogenic or likely pathogenic in a public or private genomic database. In some embodiments, the variant sequences that have been observed previously as being germline in origin and are listed as either pathogenic or likely pathogenic have been submitted to the public or private genomic database by at least two submitters. In some embodiments, the first predetermined threshold is determined empirically by calculating allele frequency values for variant sequence data comprising a known number of common germline variant sequences. In some embodiments, the first predetermined threshold is adjusted so that at least 85%, at least 90%, at least 95%, or at least 98% of the known germline variant sequences in the variant sequence data are reported as potential germline variants. In some embodiments, the first predetermined threshold is different for variant sequence data derived from different assay formats. In some embodiments, the first predetermined threshold for variant sequence data derived from a liquid biopsy assay format is larger than that for variant sequence data derived from a tissue-based assay format. In some embodiments, the first predetermined threshold ranges from about 5% to about 40%. In some embodiments, the list of gene loci further comprises a list of gene loci comprising founder mutations associated with a disease state that may be the same as or different from a known or suspected disease state for the sample. In some embodiments, the report further comprises a recommendation for further testing of the subject to confirm that the clinically-actionable potential germline pathogenic variant is a clinically-actionable germline pathogenic variant. In some embodiments, the confirmed clinically actionable germline pathogenic variant is used to diagnose or confirm a diagnosis of disease or syndrome in the subject.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flowchart for identifying and reporting potential germline pathogenic variant sequences according to the methods disclosed herein.

FIG. 2 provides a non-limiting example of a report section (or banner) for reporting potential germline pathogenic variant sequences.

FIG. 3 depicts an exemplary computing device, in accordance with some instances of the systems described herein.

FIG. 4 depicts an exemplary computer system or computer network, in accordance with some instances of the systems described herein.

### DETAILED DESCRIPTION

Disclosed herein are methods and systems for identifying and reporting clinically-actionable potential germline variants. The methods comprise identifying variant sequences (e.g., short variants) in sequence read data for selected gene loci (e.g., cancer susceptibility gene loci) that may represent actionable pathogenic germline variants, and reporting them to ensure that the healthcare provider and patient consider follow-up germline testing and/or genetic counseling. Potential germline pathogenic variants are identified by first filtering the variant sequences occurring within one of the selected gene loci for inclusion in a genomic database (e.g., the ClinVar database, National Center for Biotechnology Information, Bethesda, MD) as being pathogenic or likely pathogenic, and then filtering the results by variant allele frequency (VAF) based on a comparison to a specified assay-specific VAF threshold.

In some instances, for example, the method for reporting clinically-actionable potential germline variants may comprise: receiving, at one or more processors, variant sequence data for one or more gene loci within one or more subgenomic intervals in a sample from a subject; filtering, using the one or more processors, the variant sequence data to identify variant sequences located in a gene locus from a list of gene loci comprising clinically-actionable germline variants; filtering, using the one or more processors, the variant sequence data for variant sequences identified as being located in a gene locus from the list to identify variant sequences that are potentially germline pathogenic; filtering, using the one or more processors, the variant sequence data for variant sequences identified as being located in a gene locus from the list and as being potentially germline pathogenic to identify variant sequences that have an allele frequency greater than a first predetermined threshold; and based on a determination that a variant sequence identified as being located in a gene locus from the list and as being potentially germline pathogenic has an allele frequency greater than the first predetermined threshold, reporting the variant sequence as a clinically-actionable potential germline pathogenic variant.

In some instances, the method may further comprise making a recommendation for follow-up germline testing if a variant sequence is reported as being a clinically-actionable potential germline pathogenic variant. In some instances, the method may further comprise providing a description of one or more potential disease states that may be associated with the clinically-actionable potential germline pathogenic variant as part of the report. In some instances, the method may further comprise providing a description of one or more treatment options that may be applicable for the one or more potential disease states as part of the report. In some instances, the method may further comprise providing a description of potential disease state prevention measures for the subject or the subject's family members.

The disclosed methods may be used, for example, with nucleic acid sequencing-based pipelines for identifying variant sequences without using a paired normal sample, which is often unavailable and which, even if available, requires additional sample processing and sequencing steps which add to the overall cost of a diagnostic or prognostic procedure. As noted above, such methods may detect and report both somatic and germline variants, but often cannot distinguish between them. Algorithms for distinguishing between germline and somatic mutations are under development (see, e.g., Sun, et al. (2018), “A Computational Approach to Distinguish Somatic vs. Germline Origin of Genomic Alterations From Deep Sequencing of Cancer Specimens Without A Matched Normal”, PLOS Computational Biology 14(2):e1005965) but haven't yet been clinically validated. Some studies have reported using variant allele frequency thresholds for selected cancer susceptibility genes (CSGs) to identify potential germline variants (see, for example, Mandelker, et al. (2019), “Germline-Focussed Analysis of Tumour-Only Sequencing: Recommendations from the ESMO Precision Medicine Working Group”, Annals of Oncology 30:1221-123; and Klek, et al. (2020), “Genetic Counseling and Germline Testing in the Era of Tumor Sequencing: A Cohort Study”, JNCI Cancer Spectrum 4(3):pkaa018).

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

As used herein, the term “clinically-actionable germline pathogenic variant” refers to a variant sequence (or mutation) which is germline in origin, which is diagnostic and/or prognostic for a disease or syndrome (e.g., hereditary cancer predisposition syndrome), and for which there are known preventative, management, and/or treatment recommendations that may be applied.

The section headings used herein are for organizational purposes only and are not to be construed as limiting the subject matter described.

### Methods for Identifying and Reporting Potential Germline Pathogenic Variant Sequences:

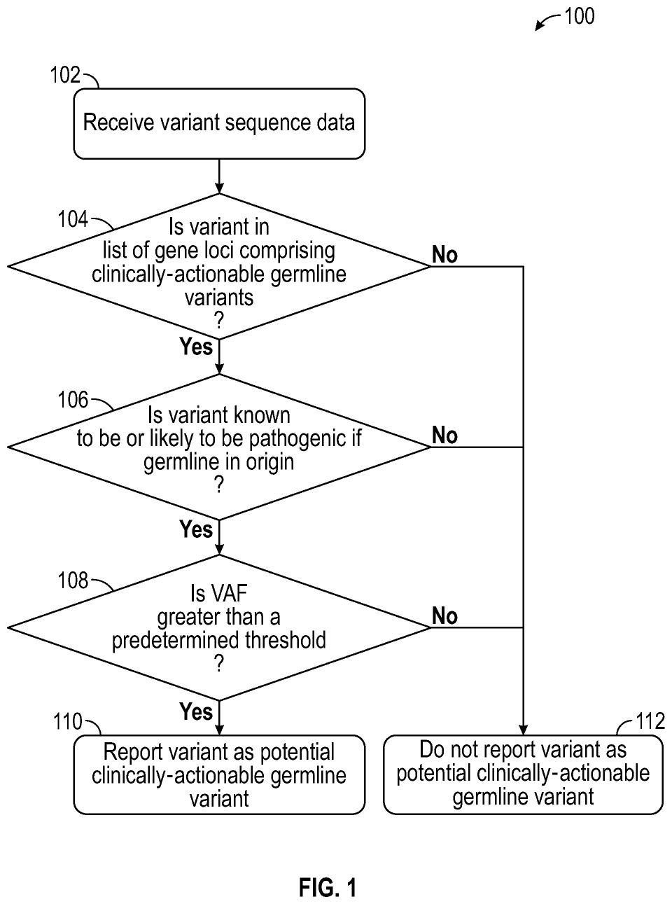

FIG. 1 provides a non-limiting example of a flowchart for a process 100 for identifying and reporting potential germline pathogenic variant sequences according to the methods disclosed herein. In some instances, the process is partially- or fully-automated. Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1, a data set comprising a list of variant sequences is received, e.g., by one or more processors used to implement the disclosed methods. In some instances, the list of variant sequences may be derived from sequence read data for a plurality of sequence reads that overlap one or more gene loci within one or more subgenomic intervals in a sample, e.g., a sample derived from a patient or other subject. In some instances, the list of variant sequences is provided by a variant calling pipeline used to process the sequence read data and identify variant sequences (e.g., short variants).

At step 104 in FIG. 1, the list of variant sequences is filtered to identify those variant sequences that are located in a gene locus known to comprise clinically-actionable germline variants. If the variant sequence is not located within a gene locus from the list of selected gene loci, it is added to a list of variants that will not be reported at step 112.

In some instances, the list of gene loci is selected from a source of genomic information. In some instances, for example, the source of genomic information comprises a public or private genomic database. In some instances, the list of gene loci may comprise, for example, a list of cancer susceptibility genes that are known to comprise clinically-actionable germline variants. In some instances, the list of gene loci may comprise a list of cancer susceptibility genes that is based on a published set of professional guidelines (e.g., the ESMO Precision Medicine Working Group guidelines (Mandelker, et al. (2019), “Germline-Focused Analysis of Tumour-Only Sequencing: Recommendations from the ESMO Precision Medicine Working Group”, Annals of Oncology 30:1221-123)). In some instances, the list may comprise cancer susceptibility gene loci for which variant sequences have a probability of greater than a predetermined probability threshold of being germline in origin if identified during tumor sample sequence. For example, in some instances, the predetermined threshold may range from about 5% to about 20%. In some instances, the predetermined probability threshold may be 5%, 10%, 15%, or 20%. In some instances, the predetermined probability threshold may be at least 5%, at least 10%, at least 15%, or at least 20%.

In some instances, the list of gene loci comprises the ATM, BAP1, BRCA1, BRCA2, BRIP1, CHEK2, FH, FLCN, MLH1, MSH2, MSH6, MUTYH, PALB2, PMS2, POLE, RAD51C, RAD51D, RET, SDHA, SDHB, SDHC, SDHD, TSC2, VHL gene loci, or any combination thereof.

In some instances, the list of gene loci may further comprise a list of gene loci comprising founder mutations associated with a disease state that may be the same as or different from a known or suspected disease state for the sample.

In some instances, the method may additionally or alternatively comprise taking into account the tumor fraction of a liquid biopsy, or the tumor purity of a tissue sample, for identifying potential germline variants. Tumor fraction or tumor purity for a sample may be calculated, e.g., from sequencing data, or estimated (for tissue samples) based on a review by a pathologist or using a digital pathology algorithm. See, for example, Sun, et al. (2018), “A Computational Approach to Distinguish Somatic vs. Germline Origin of Genomic Alterations from Deep Sequencing of Cancer Specimens Without a Matched Normal”, PLOS Computational Biology 14(2):e1005965, which provides a non-limiting example of a computational approach to identifying variant sequences that are of germline origin.

At step 106 in FIG. 1, the subset of variant sequences that are located within a gene locus in the list of gene loci are additionally filtered to determine if the variant sequence is known to be, or is likely to be, pathogenic if germline in origin. If it is not, then the variant sequence is added to a list of variants that will not be reported at step 112.

In some instances, variant sequences located in a gene locus on the list may be filtered to identify those variant sequences that are potentially germline pathogenic, e.g., based on a sequence comparison to known clinically-actionable germline variants for the gene locus. In some instances, the identification of a variant sequence as being potentially germline pathogenic may comprise identifying the variant sequence as being listed as potentially germline pathogenic in a public or private genomic database. In some instances, the variant sequence may be identified as potentially germline pathogenic if it has been observed previously as germline and is listed as either pathogenic or likely pathogenic in the public or private genomic database. In some instances, the variant sequence may be identified as potentially germline pathogenic if it is listed as either pathogenic or likely pathogenic, and has been submitted to the public or private genomic database by at least two submitters (e.g., two different submitters).

At step 108 in FIG. 1, the subset of variant sequences that are both located within a gene locus in the list of gene loci known to be, or likely to be, pathogenic if germline in origin are additionally filtered to determine if their variant allele frequency (VAF) is greater than a predetermined VAF threshold in the sample (i.e., the variant must be detected at a VAF that is biologically plausible to be of germline origin). If so, then the variant sequence is reported as a clinically-actionable potential germline variant at step 110. If not, then the variant sequence is added to a list of variants that will not be reported at step 112.

In some instances, the predetermined VAF threshold is determined or adjusted empirically by calculating the VAF values of a known number of common germline variant sequences. In some instances, for example, the predetermined VAF threshold may be adjusted so that at least 85%, at least 90%, at least 95%, or at least 98% of true germline variant sequences in the variant sequence data are reported as potential germline variants.

In some instances, the predetermined VAF threshold may be different for variant sequence data derived from different assay formats. For example, the predetermined VAF threshold for variant sequence data derived from a liquid biopsy assay format may be different (e.g., larger) than that for variant sequence data derived from a tissue-based assay format. In some instances, e.g., for a liquid biopsy assay format or for a tissue-based assay format, the predetermined VAF threshold may range from about 5% to about 40%. In some instances, e.g., for a liquid biopsy assay format or for a tissue-based assay format, the predetermined VAF threshold may be 5%, 10%, 15%, 20%, 25%, 30%, 35%, or 40%. In some instances, e.g., for a liquid biopsy assay format or for a tissue-based assay format, the predetermined VAF threshold may be at least 5%, at least 10%, at least 15%, at least 20%, at least 25%, at least 30%, at least 35%, or at least 40%.

In some instances, process 100 depicted in FIG. 1 may further comprise making a recommendation for follow-up germline testing if a variant sequence is reported as being a clinically-actionable potential germline pathogenic variant. In some instances, process 100 may further comprise providing a description of one or more potential disease states that may be associated with the clinically-actionable potential germline pathogenic variant as part of the report. In some instances, process 100 may further comprise providing a description of one or more treatment options that may be applicable for the one or more potential disease states as part of the report. In some instances, process 100 may further comprise providing a description of potential disease state prevention measures for the subject or the subject's family members.

In some instances, process 100 may further comprise identifying and reporting germline passenger mutations (e.g., mutations that do not directly drive cancer initiation and progression) that are associated with a clinically-actionable potential germline pathogenic variant. In some instances, the presence of germline passenger mutations may impact a treatment decision.

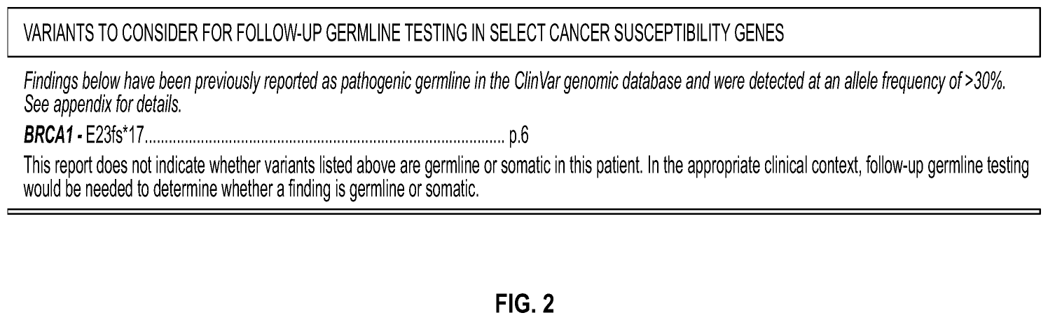

FIG. 2 provides a non-limiting example of a report section (or banner) for reporting potential germline pathogenic variant sequences that may be output by the disclosed methods. In some instances, the report (or banner) may comprise a listing of specific potential germline pathogenic variants identified, the gene loci in the list of gene loci, a listing of the criteria used to identify the potential germline pathogenic variants (e.g., the genomic database used to identify potentially pathogenic germline variants, the professional guideline(s) used, etc.), the variant allele frequencies for the identified potential germline pathogenic variants, the variant allele frequency threshold used to discriminate between reported potential germline pathogenic variants and variant sequences not reported as potential germline, or any combination thereof.

As noted above, in some instances, the report may further comprise (as part of the banner section, or in another report section such as an appendix) a recommendation for follow-up germline testing, a description of one or more potential disease states that may be associated with the clinically-actionable potential germline pathogenic variant, a description of one or more treatment options that may be applicable for the one or more potential disease states, a description of potential disease state prevention measures for the subject or the subject's family members, or any combination thereof.

In some instances, the disclosed methods may be used to identify potential germline pathogenic variant sequence in at least 1, at least 2, at least 3, at least 4, at least 5, at least 6, at least 7, at least 8, at least 9, at least 10, at least 20, at least 30, at least 40, or more than 40 gene loci.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for identifying potential germline pathogenic variant sequences may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for identifying potential germline pathogenic variant sequences and/or passenger mutations may be used to predict genetic disorders in fetal DNA. (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify potential germline pathogenic variants.

In some instances, the disclosed methods for identifying potential germline pathogenic variant sequences may be used to screen and/or select a subject (e.g., a patient) for a clinical trial based on the detection of potential germline pathogenic variants in one or more gene loci. In some instances, patient selection for clinical trials based on, e.g., identification of potential germline pathogenic variants in one or more gene loci, may accelerate the development of targeted therapies and improve the healthcare outcomes for treatment decisions.

In some instances, the disclosed methods for identifying potential germline pathogenic variant sequences may be used to select (or as part of a selection of) an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the disclosed methods for identifying potential germline pathogenic variant sequences may be used in treating a disease (e.g., a cancer) in a subject. For example, in response to determining that one or more potential germline mutations are present, an effective amount of an anti-cancer therapy or anti-cancer treatment may be administered to the subject.

In some instances, the disclosed methods for identifying potential germline pathogenic variants may be used for monitoring disease progression or recurrence (e.g., cancer or tumor progression or recurrence) in a subject. For example, in some instances, the methods may be used to detect potential germline pathogenic variants and/or associated passenger mutations in a first sample obtained from the subject at a first time point, and used to detect potential germline pathogenic variants and/or associated passenger mutations in a second sample obtained from the subject at a second time point, where comparison of the first determination and the second determination allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the detected potential germline pathogenic variants (or allele frequencies thereof).

In some instances, the presence of potential germline pathogenic variants determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for identifying and reporting potential germline pathogenic variants may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for identifying and reporting potential germline pathogenic variants as part of a genomic profiling process (or inclusion of the output from the disclosed methods for identifying and reporting potential germline pathogenic variants as part of the genomic profile of the subject) can improve the validity of, e.g., disease detection calls and treatment decisions, made on the basis of the genomic profile by, for example, independently confirming the presence of potential germline pathogenic variants in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

In some instances, as described elsewhere herein, the disclosed methods for identifying potential germline pathogenic variant sequences may be used to flag patients and their family members for potential referral to genetic counseling and/or dedicated germline testing.

### Samples

The disclosed methods and systems may be used with any of a variety of samples (also referred to herein as specimens) comprising nucleic acids (e.g., DNA or RNA) that are collected from a subject (e.g., a patient). Examples include, but are not limited to, a tumor sample, a tissue sample, a biopsy sample, a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavages or bronchoalveolar lavages), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly (A) tail of mature mRNAs by priming with oligo (dT)-containing oligonucleotides. Methods for depletion, poly (A) enrichment, and cDNA synthesis are well known to those of skill in the art.

In some instances, the sample may comprise a tumor content, e.g., comprising tumor cells or tumor cell nuclei. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g. a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164 (1): 35-42; Masuda, et al., (1999) Nucleic Acids Res. 27 (22): 4436-4443; Specht, et al., (2001) Am J Pathol. 158 (2): 419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4 (11): 903-5; Hodges, E. et al. (2007) Nat. Genet. 39 (12): 1522-7; and Okou, D. T. et al. (2007) Nat. Methods 4 (11): 907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing”, and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 103, 104, 105 or more than 105 molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S.L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147 (1): 195-197), the Striped Smith-Waterman algorithm (see, e.g., Farrar (2007), “Striped Smith-Waterman Speeds Database Searches Six Times Over Other SIMD Implementations”, Bioinformatics 23 (2): 156-161), the Needleman-Wunsch algorithm (Needleman, et al. (1970) “A General Method Applicable to the Search for Similarities in the Amino Acid Sequence of Two Proteins”, J. Molecular Biology 48 (3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

A Bayesian mutation-detection approach compares the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9): 1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25 (16): 2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26 (6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Systems

Also disclosed herein are systems designed to implement any of the disclosed methods for identifying and reporting potential germline pathogenic variants in a sample from a subject. The systems may comprise, e.g., one or more processors, and a memory unit communicatively coupled to the one or more processors and configured to store instructions that, when executed by the one or more processors, cause the system to: receive, at one or more processors, variant sequence data for one or more gene loci within one or more subgenomic intervals in a sample from a subject; filter, using the one or more processors, the variant sequence data to identify variant sequences located in a gene locus from a list of gene loci comprising clinically-actionable germline variants; filter, using the one or more processors, the variant sequence data for variant sequences identified as being located in a gene locus from the list to identify variant sequences that are potentially germline pathogenic; filter, using the one or more processors, the variant sequence data for variant sequences identified as being located in a gene locus from the list and as being potentially germline pathogenic to identify variant sequences that have an allele frequency greater than a first predetermined threshold; and based on a determination that a variant sequence identified as being located in a gene locus from the list and as being potentially germline pathogenic has an allele frequency greater than the first predetermined threshold, report the variant sequence as a clinically-actionable potential germline pathogenic variant.

In some instances, the disclosed systems may further comprise a sequencer, e.g., a next generation sequencer (also referred to as a massively parallel sequencer). Examples of next generation (or massively parallel) sequencing platforms include, but are not limited to, the Roche 454, Illumina Solexa, ABI-SOLID, ION Torrent, or Pacific Bioscience sequencing platforms.

In some instances, the disclosed systems may be used for identifying and reporting potential germline pathogenic variant sequences in any of a variety of samples as described herein (e.g., a tissue sample, biopsy sample, hematological sample, or liquid biopsy sample derived from the subject).

In some instances, the plurality of gene loci for which sequencing data is processed to identify and report the presence of potential germline pathogenic variant sequences may comprise at least 1, 2, 3, 4, 5, 6, 7, 8, 9, 10, or more than 10 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the identification of potential germline pathogenic variants is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

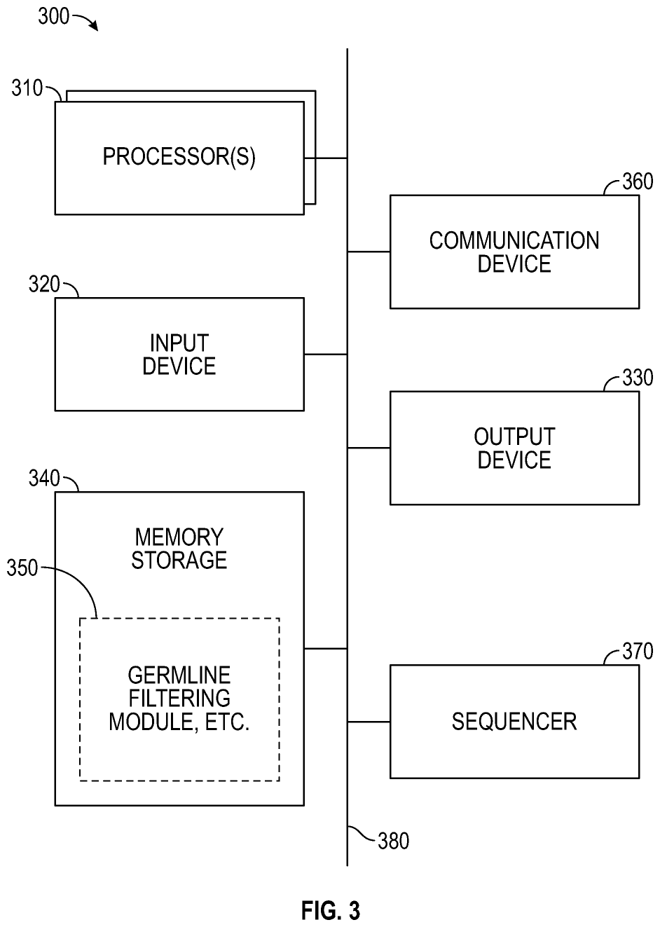

FIG. 3 illustrates an example of a computing device or system in accordance with one embodiment. Device 300 can be a host computer connected to a network. Device 300 can be a client computer or a server. As shown in FIG. 3, device 300 can be any suitable type of microprocessor-based device, such as a personal computer, workstation, server or handheld computing device (portable electronic device) such as a phone or tablet. The device can include, for example, one or more processor(s) 310, input devices 320, output devices 330, memory or storage devices 340, communication devices 360, and nucleic acid sequencers 370. Software 350 residing in memory or storage device 340 may comprise, e.g., an operating system as well as software for executing the methods described herein. Input device 320 and output device 330 can generally correspond to those described herein, and can either be connectable or integrated with the computer.

Input device 320 can be any suitable device that provides input, such as a touch screen, keyboard or keypad, mouse, or voice-recognition device. Output device 330 can be any suitable device that provides output, such as a touch screen, haptics device, or speaker.

Storage 340 can be any suitable device that provides storage (e.g., an electrical, magnetic or optical memory including a RAM (volatile and non-volatile), cache, hard drive, or removable storage disk). Communication device 360 can include any suitable device capable of transmitting and receiving signals over a network, such as a network interface chip or device. The components of the computer can be connected in any suitable manner, such as via a wired media (e.g., a physical system bus 380, Ethernet connection, or any other wire transfer technology) or wirelessly (e.g., Bluetooth®, Wi-Fi®, or any other wireless technology).

Software module 350, which can be stored as executable instructions in storage 340 and executed by processor(s) 310, can include, for example, an operating system and/or the processes that embody the functionality of the methods of the present disclosure (e.g., as embodied in the devices as described herein).

Software module 350 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 340, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 350 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

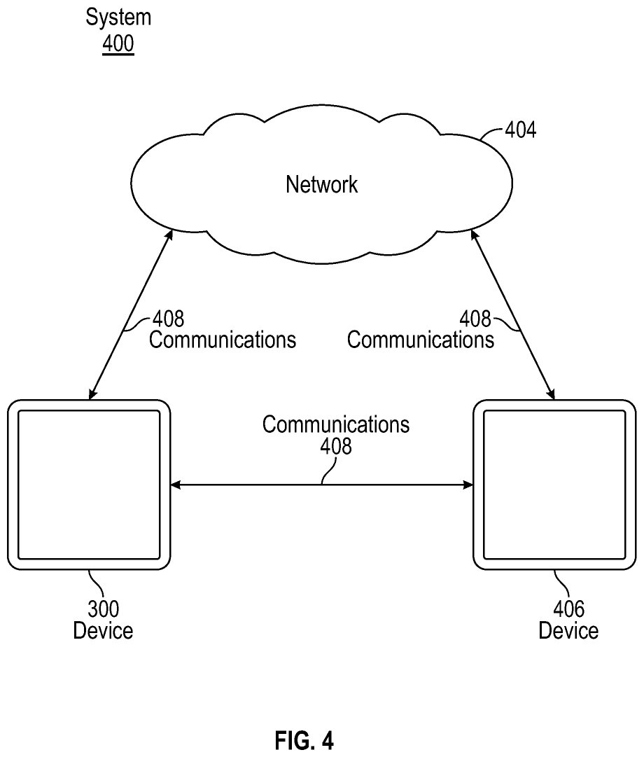

Device 300 may be connected to a network (e.g., network 404, as shown in FIG. 4 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 300 can be implemented using any operating system, e.g., an operating system suitable for operating on the network. Software module 350 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 310.

Device 300 can further include a sequencer 370, which can be any suitable nucleic acid sequencing instrument.

FIG. 4 illustrates an example of a computing system in accordance with one embodiment. In system 400, device 300 (e.g., as described above and illustrated in FIG. 3) is connected to network 404, which is also connected to device 406. In some embodiments, device 406 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio RS system.

Devices 300 and 406 may communicate, e.g., using suitable communication interfaces via network 404, such as a Local Area Network (LAN), Virtual Private Network (VPN), or the Internet. In some embodiments, network 404 can be, for example, the Internet, an intranet, a virtual private network, a cloud network, a wired network, or a wireless network. Devices 300 and 406 may communicate, in part or in whole, via wireless or hardwired communications, such as Ethernet, IEEE 802.11b wireless, or the like. Additionally, devices 300 and 406 may communicate, e.g., using suitable communication interfaces, via a second network, such as a mobile/cellular network. Communication between devices 300 and 406 may further include or communicate with various servers such as a mail server, mobile server, media server, telephone server, and the like. In some embodiments, Devices 300 and 406 can communicate directly (instead of, or in addition to, communicating via network 404), e.g., via wireless or hardwired communications, such as Ethernet, IEEE 802.11b wireless, or the like. In some embodiments, devices 300 and 406 communicate via communications 408, which can be a direct connection or can occur via a network (e.g., network 404).

One or all of devices 300 and 406 generally include logic (e.g., http web server logic) or are programmed to format data, accessed from local or remote databases or other sources of data and content, for providing and/or receiving information via network 404 according to various examples described herein.

### EXAMPLES

### Example 1-Detection of a Potential PALB2 Germline Pathogenic Variant

This section provides a non-limiting example of the identification and reporting of a PALB2 germline pathogenic variant. A variant in the PALB2 gene is detected in a patient's sample using a genomic profiling assay. This variant has been previously identified as a pathogenic germline variant in a publicly available genomic database (e.g., the ClinVar database), and is identified in the patient's sample at an allele frequency of 35%. Because this variant meets the criteria developed to identify actionable potential germline variants (present in one of a list of selected gene loci, listed as pathogenic or likely pathogenic in a genomic database, and exhibiting a variant allele frequency greater than a predetermined VAF threshold of, e.g., about 10%) according to the disclosed methods, this variant is included in the germline banner in a report to flag the physician to consider follow-up confirmatory germline testing.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
receiving, at one or more processors, sequence read data for the plurality of sequence reads;
identifying, using the one or more processors, variant sequence data for one or more gene loci within the sequence read data for the plurality of sequence reads;
filtering, using the one or more processors, the variant sequence data to identify variant sequences located in a gene locus from a list of gene loci comprising clinically-actionable germline variants;
filtering, using the one or more processors, the variant sequences located in a gene locus on the list to identify variant sequences that are potentially germline pathogenic based on a sequence comparison to known clinically-actionable germline variants for the gene locus;
filtering, using the one or more processors, the potentially pathogenic germline variant sequences to identify variant sequences that have an allele frequency greater than a first predetermined threshold; and
based on a determination that a particular potentially pathogenic germline variant sequence has an allele frequency greater than the first predetermined threshold, identifying the variant sequence as a clinically-actionable potential germline pathogenic variant.

2.-29. (canceled)

30. The method of claim 1, further comprising generating a report that identifies the variant sequence as a clinically-actionable potential germline pathogenic variant.

31. The method of claim 30, further comprising generating a recommendation for follow-up germline testing if a variant sequence is reported as being a clinically-actionable potential germline pathogenic variant, and including the recommendation in the report.

32.-34. (canceled)

35. The method of claim 1, wherein the list of gene loci is selected from a source of genomic information and wherein the source of genomic information comprises a public or private genomic database.

36. (canceled)

37. The method of claim 1, wherein the list of gene loci comprises a list of cancer susceptibility genes that comprise clinically-actionable germline variants and wherein the list of cancer susceptibility genes comprises a list of gene loci for which variant sequences have a probability of greater than a second predetermined threshold of being germline in origin if identified during tumor sample sequence.

38. (canceled)

39. The method of claim 37, wherein the second predetermined threshold ranges from 5% to 20%.

40. (canceled)

41. The method of claim 1, further comprising identifying and reporting germline passenger mutations associated with a clinically-actionable potential germline pathogenic variants.

42. The method of claim 1, wherein the known clinically-actionable germline variants for the gene locus comprise variant sequences that have been observed previously as being germline in origin and are listed as either pathogenic or likely pathogenic in a public or private genomic database.

43. (canceled)

44. The method of claim 1, wherein the first predetermined threshold is determined empirically by calculating allele frequency values for variant sequence data comprising a known number of common germline variant sequences.

45. The method of claim 44, wherein the first predetermined threshold is adjusted so that at least 85%, at least 90%, at least 95%, or at least 98% of the known germline variant sequences in the variant sequence data are reported as potential germline variants.

46. The method of claim 1, wherein the first predetermined threshold is different for variant sequence data derived from different assay formats and wherein the first predetermined threshold for variant sequence data derived from a liquid biopsy assay format is larger than that for variant sequence data derived from a tissue-based assay format.

47. (canceled)

48. The method of claim 1, wherein the first predetermined threshold ranges from about 5% to about 40%.

49. The method of claim 1, wherein the list of gene loci further comprises a list of gene loci comprising founder mutations associated with a disease state that may be the same as or different from a known or suspected disease state for the sample.

50. The method of claim 1, further comprising testing the subject to confirm that the clinically-actionable potential germline pathogenic variant is a clinically-actionable germline pathogenic variant.

51. The method of claim 50, further comprising using the confirmed clinically actionable germline pathogenic variant to diagnose or confirm a diagnosis of disease or syndrome in the subject.

52. The method of claim 51, wherein the disease is cancer.

53. (canceled)

54. (canceled)

55. The method of claim 51, further comprising selecting an anti-cancer therapy to administer to the subject based on the confirmed clinically-actionable germline pathogenic variant.

56. The method of claim 55, further comprising determining an effective amount of the anti-cancer therapy to administer to the subject and administering the anti-cancer therapy to the subject.

57.-67. (canceled)

68. A system comprising:
one or more processors; and
a memory communicatively coupled to the one or more processors and configured to store instructions that, when executed by the one or more processors, cause the system to:
receive variant sequence data for one or more gene loci within one or more subgenomic intervals in a sample from a subject;
filter the variant sequence data to identify variant sequences located in a gene locus from a list of gene loci comprising clinically-actionable germline variants;
filter the variant sequences located in a gene locus on the list to identify variant sequences that are potentially germline pathogenic based on a sequence comparison to known clinically-actionable germline variants for the gene locus;
filter the potentially germline pathogenic variant sequences to identify variant sequences that have an allele frequency greater than a first predetermined threshold; and
based on a determination that a particular potentially pathogenic germline variant sequence has an allele frequency greater above the first predetermined threshold, report the potentially pathogenic germline variant sequence as a clinically-actionable potential germline pathogenic variant.

69.-97. (canceled)

98. A method for reporting clinically-actionable potential germline variants comprising:
receiving, at one or more processors, variant sequence data for one or more gene loci in a sample from a subject;
filtering, using the one or more processors, the variant sequence data to identify variant sequences located in a gene locus from a list of gene loci comprising clinically-actionable germline variants;
filtering, using the one or more processors, the variant sequences located in a gene locus on the list to identify variant sequences that are potentially germline pathogenic based on a sequence comparison to known clinically-actionable germline variants for the gene locus;
filtering, using the one or more processors, the potentially germline pathogenic variant sequences to identify variant sequences that have an allele frequency greater than a first predetermined threshold; and
based on a determination that a particular potentially pathogenic germline variant sequence has an allele frequency above the first predetermined threshold, reporting the potentially pathogenic germline variant sequence as a clinically-actionable potential germline pathogenic variant.
